(19) United States
(12) Patent Application Publication
(10) Pub. No.: US 2021/0052483 A1
(43) Pub. Date: Feb. 25, 2021

(54) OILY COSMETIC AND METHOD FOR PRODUCING THE SAME

(71) Applicant: SHISEIDO COMPANY, LTD., TOKYO (JP)

(72) Inventors: SHUN KUBOTA, KANAGAWA (JP); Tomo OSAWA, KANAGAWA (JP); Shoji TAJIMA, KANAGAWA (JP); Motoharu KIMURA, KANAGAWA (JP)

(73) Assignee: SHISEIDO COMPANY, LTD., TOKYO (JP)

(21) Appl. No.: 16/964,618

(22) PCT Filed: Jan. 23, 2019
(86) PCT No.: PCT/JP2019/002018

§ 371 Date: Jul. 24, 2020

(30) Foreign Application Priority Data
Feb. 5, 2018 (JP) 2018-018191

(51) Int. Cl. A61K 8/891; A61K 8/92; A61Q 17/04

(52) U.S. Cl. CPC A61K 8/891; A61Q 17/04; A61K 8/92

(57) ABSTRACT

The oily cosmetic includes 4 to 22 mass % of a polyether modified silicone wax and 40 to 80 mass % of an oily component. The oily component contains 46 mass % or more of a volatile oily component. The volatile oily component is contained in an amount of 25 mass % or more with respect to the mass of the oily cosmetic.

### CROSS REFERENCE TO RELATED APPLICATIONS

The present invention is based upon and claims the benefit of the priority of Japanese Patent Application No. 2018-18191 (filed on Feb. 5, 2018), the disclosure of which is incorporated herein in its entirety by reference.

### TECHNICAL FIELD

The present disclosure relates to an oily cosmetic and a method for producing the same.

### BACKGROUND ART

Cosmetics including, in combination, a silicone compound and a thickener to improve the feel upon use are known in the art (see, for example, Patent Literature 1). The water-based skin cosmetic disclosed in Patent Literature 1 includes a silicone compound represented by a specific structural formula, and a thickener constituted by a microgel prepared according to a specific production method.

### CITATION LIST

### Patent Literature

Patent Literature 1: Japanese Unexamined Patent Publication No. 2013-112678A

### SUMMARY OF INVENTION

### Technical Problem

Skin made up with an oily cosmetic has low wettability to water. Thus, sweat tends to form beads on the makeup. Bead-like sweat has a small surface area and is thus less prone to evaporate. Bead-like sweat formed on the face without evaporating is likely to trickle down, and trickling sweat causes makeup smudging. Wiping sweat with a towel etc. to prevent sweat from trickling down also causes makeup smudging. Further, from an aesthetic viewpoint, women in particular tend to dislike it when beads of sweat form on the face. Thus, there is a demand for an oily cosmetic capable of working against sweat.

Water-based cosmetics, such as the one disclosed in Patent Literature 1, have greater affinity to sweat compared to oily cosmetics, and thus tend to smudge easily.

Further, the silicone compound blended to the water-based skin cosmetic disclosed in Patent Literature 1 is hydrophilic; thus, the silicone compound is difficult to disperse and blend uniformly in an oily cosmetic, even when attempts are made to blend it into the oily cosmetic. Thus, there is a demand for a production method for uniformly blending a hydrophilic silicone wax into an oily cosmetic.

### Solution to Problem

According to a first aspect of the present disclosure, an oily cosmetic is provided, the oily cosmetic comprising from 4 to 22% by mass of a polyether-modified silicone wax, and from 40 to 80% by mass of an oily component. At least 46% by mass of the oily component is a volatile oily component. A content by percentage of the volatile oily component is at least 25% by mass relative to the mass of the oily cosmetic.

According to a second aspect of the present disclosure, an oily cosmetic production method is provided, the method comprising dissolving a hydrophilic polyether-modified silicone wax in a first oily component to prepare a solution, and mixing the solution and a second oily component.

### Advantageous Effects of Invention

According to the oily cosmetic of the present disclosure, it is possible to suppress formation of bead-like sweat in regions where the cosmetic has been applied. Thus, drying of sweat can be promoted. It is also possible to suppress makeup smudging caused by trickling-down of sweat and/or wiping of sweat. Further, it is possible to make sweat look less conspicuous.

According to the oily cosmetic production method of the present disclosure, it is possible to obtain an oily cosmetic in which a hydrophilic silicone wax is blended uniformly.

### BRIEF DESCRIPTION OF DRAWINGS

FIG. 1 is a schematic flowchart of a method for producing an oily cosmetic according to a second embodiment.

FIG. 2 is a photograph of an upper part of the face when the user has applied cosmetics according to Test Examples 1 and 2 and sweated.

FIG. 3 is a photograph of a lower part of the face when the user has applied cosmetics according to Test Examples 1 and 2 and sweated.

### DESCRIPTION OF EMBODIMENTS

According to a preferred mode of the above first aspect, the volatile oily component includes a silicone oil.

According to a preferred mode of the above first aspect, the oily cosmetic further comprises from 10 to 40% by mass of a powder relative to the mass of the oily cosmetic.

According to a preferred mode of the above first aspect, the powder includes an oil-absorbent powder.

According to a preferred mode of the above first aspect, a content by percentage of water is 5% by mass or less.

According to a preferred mode of the above first aspect, the polyether-modified silicone wax includes a compound represented by Chem. 1 below.

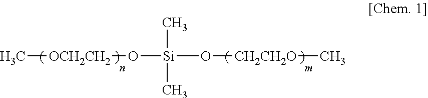

(wherein m and n each independently represent an integer from 10 to 24).

According to a preferred mode of the above first aspect, the polyether-modified silicone wax includes bis-PEG-18 methyl ether dimethyl silane.

According to a preferred mode of the above first aspect, the oily component includes at least one of an ester oil and/or a non-volatile silicone oil.

According to a preferred mode of the above first aspect, the oily component includes a UV absorber.

According to a preferred mode of the above first aspect, the proportion of the volatile oily component within the oily component is from 48 to 74% by mass relative to the mass of the oily component. The content by percentage of the volatile oily component is from 28 to 46% by mass relative to the mass of the oily cosmetic.

According to a preferred mode of the above second aspect, the first oily component includes a UV absorber.

According to a preferred mode of the above second aspect, the first oily component includes at least one compound selected from the group consisting of 2-ethylhexyl 4-methoxycinnamate, octocrylene, diethylamino hydroxybenzoyl hexyl benzoate, butyl methoxydibenzoylmethane, ethylhexyl triazone, and bis-ethylhexyloxyphenol methoxyphenyl triazine.

According to a preferred mode of the above second aspect, the second oily component includes a volatile oily component.

According to a preferred mode of the above second aspect, the volatile oily component includes a silicone oil.

According to a preferred mode of the above second aspect, the polyether-modified silicone wax includes a compound represented by Chem. 2 below.

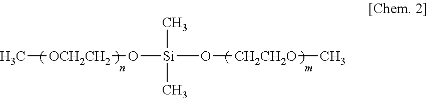

(wherein m and n each independently represent an integer from 10 to 24).

According to a preferred mode of the above second aspect, the polyether-modified silicone wax includes bis-PEG-18 methyl ether dimethyl silane.

An oily cosmetic according to a first embodiment of the present disclosure is described. The oily cosmetic includes a polyether-modified silicone wax and an oily component.

{Polyether-Modified Silicone Wax}

For the polyether-modified silicone wax, it is possible to use, for example, a polyoxyethylene-modified silicone wax. For the polyoxyethylene-modified silicone wax, it is possible to use a compound represented by Chem. 3 below.

By using a polyoxyethylene-modified silicone wax, particularly a compound represented by Chem. 3 below, wettability to water can be improved. Thus, it is possible to suppress formation of bead-like sweat on makeup applied to the skin. That is, sweat can be spread out thinly along the makeup surface (skin surface) so as to increase the surface area.

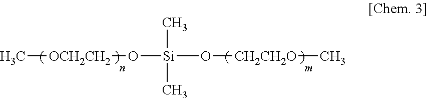

In Chem. 3 above, m and n may each independently represent an integer from 10 to 24; m and n may be different numbers or the same number. For the silicone wax, it is possible to use, for example, bis-PEG-18 methyl ether dimethyl silane wherein m and n each represent an average of 18. An example of a commercially available product of bis-PEG-18 methyl ether dimethyl silane may include Dow Corning (registered trademark) 2501 Cosmetic Wax (from Dow Corning Toray Co., Ltd.).

The content by percentage of the silicone wax relative to the mass of the oily cosmetic is preferably 4% by mass % by mass or greater, more preferably 5% by mass % by mass or greater. If the content by percentage of the silicone wax is less than 4% by mass, the effect of improving wettability to sweat cannot be achieved sufficiently. To further improve quick-drying of sweat, the content by percentage of the silicone wax relative to the mass of the oily cosmetic is preferably 7% by mass or greater, more preferably 10% by mass or greater, more preferably 13% by mass or greater, more preferably 15% by mass or greater, even more preferably 17% by mass or greater. The content by percentage of the silicone wax relative to the mass of the oily cosmetic is preferably 22% by mass or less, more preferably 20% by mass or less. If the content by percentage of the silicone wax exceeds 22% by mass, makeup smudging becomes likely. To further suppress makeup smudging, the content by percentage of the silicone wax relative to the mass of the oily cosmetic is preferably 18% by mass or less, more preferably 15% by mass or less, more preferably 13% by mass or less, more preferably 10% by mass or less, even more preferably 8% by mass or less.

{Oily Component}

For the oily component used in the oily cosmetic of the present disclosure, it is possible to use a component generally used in external skin preparations and cosmetic products. Concrete examples may include liquid oils, solid fats, waxes, hydrocarbons, higher fatty acids, higher alcohols, synthetic ester oils, and silicone oils. Note that, in the present Description, the term “oily component” encompasses oil components and components soluble in oil components.

The oily component includes a volatile oily component. In the present disclosure, the term “volatile” means that a component can easily volatilize at room temperature, outside air temperature, or body temperature, when applied to the skin. For the volatile oily component, it is possible to use, for example, a silicone oil or a hydrocarbon oil. For the volatile silicone oil, it is possible to use, for example, a silicone oil having a dimethyl polysiloxane structure. Examples of the volatile hydrocarbon oil may include isododecane, isohexadecane, and isodecane.

When the oily cosmetic is applied to the skin, a large portion of the volatile oily component within the oily cosmetic volatilizes. It is considered that, when the volatile oily component volatilizes, sections where the volatile oily component was present become voids, which serve as passages for sweat. So, in order to facilitate diffusion of sweat on the oily cosmetic, the content by percentage of the volatile oily component relative to the mass of the oily cosmetic is preferably 25% by mass or greater, more preferably 28% by mass or greater, even more preferably 30% by mass or greater. If the content of the volatile oily component is less than 25% by mass, quick-dryability and makeup durability, as well as usability, will deteriorate. The content by percentage of the volatile oily component relative to the mass of the oily cosmetic may be 35% by mass or greater, 40% by mass or greater, or 45% by mass or greater. When the content by percentage of the volatile oily component relative to the mass of the oily cosmetic is, at any rate, 50% by mass or less, sufficient quick-dryability and makeup durability can be maintained. To ensure usability in addition to quick-dryability and makeup durability, the content by percentage of the volatile oily component relative to the mass of the oily cosmetic is preferably 48% by mass or less, more preferably 46% by mass or less, more preferably 44% by mass or less, even more preferably 42% by mass or less. If the content of the volatile oily component exceeds 48% by mass, drying becomes slow when applied to the skin, thereby deteriorating usability. The content by percentage of the volatile oily component relative to the mass of the oily cosmetic may be 40% by mass or less, 35% by mass or less, or 30% by mass or less.

To ensure quick-dryability and makeup durability, the content of the volatile oily component, within the oily component(s), relative to the mass of the entirety of the oily component(s) is preferably 46% by mass or greater, more preferably 48% by mass or greater, more preferably 50% by mass or greater, more preferably 52% by mass or greater, even more preferably 54% by mass or greater. Depending on the desired blend, the content of the volatile oily component, within the oily component(s), relative to the mass of the oily component(s) may be 80% by mass or less, 75% by mass or less, or 70% by mass or less.

To improve usability in addition to quick-dryability and makeup durability, the content of the volatile oily component, within the oily component(s), relative to the mass of the entirety of the oily component(s) is preferably 46% by mass or greater, more preferably 48% by mass or greater, more preferably 50% by mass or greater, more preferably 52% by mass or greater, even more preferably 54% by mass or greater. The content of the volatile oily component, within the oily component(s), relative to the mass of the entirety of the oily component(s) is preferably 74% by mass or less, more preferably 72% by mass or less, more preferably 70% by mass or less, more preferably 68% by mass or less, even more preferably 66% by mass or less.

The oily cosmetic may include a non-volatile oily component.

The oily cosmetic of the present disclosure preferably includes a UV absorber. Among the compounds described as examples below, the UV absorber is preferably a UV absorber having a benzene skeleton. By using such a UV absorber, the hydrophilic silicone wax can be dispersed uniformly in the oily cosmetic. Preferably, the UV absorber is, for example, at least one compound selected from the group consisting of 2-ethylhexyl 4-methoxycinnamate, octocrylene, diethylamino hydroxybenzoyl hexyl benzoate, butyl methoxydibenzoylmethane, ethylhexyl triazone, and bis-ethylhexyloxyphenol methoxyphenyl triazine.

Examples of the liquid oil that may be used may include avocado oil, camellia oil, turtle oil, macadamia nut oil, corn oil, mink oil, olive oil, rapeseed oil, egg yolk oil, sesame oil, par chic oil, wheat germ oil, southern piece oil, castor oil, linseed oil, safflower oil, cotton seed oil, perilla oil, soybean oil, groundnut oil, brown real oil, torreya oil, rice bran oil, Chinese tung oil, Japanese tung oil, jojoba oil, germ oil, triglycerol, and the like.

Examples of the solid fat that may be used may include cacao butter, coconut oil, horse fat, hydrogenated coconut oil, palm oil, beef tallow, sheep tallow, hydrogenated beef tallow, palm kernel oil, lard, beef bones fat, Japan wax kernel oil, hardened oil, hoof oil, Japan wax, hydrogenated caster oil, and the like.

Examples of the waxes that may be used may include beeswax, candelilla wax, cotton wax, carnauba wax, bayberry wax, insect wax, spermaceti, montan wax, bran wax, lanolin, kapok wax, lanolin acetate, liquid lanolin, sugarcane wax, lanolin fatty acid isopropyl ester, hexyl laurate, reduced lanolin, jojoba wax, hardened lanolin, shellac wax, POE lanolin alcohol ether, POE lanolin alcohol acetate, POE cholesterol ether, lanolin fatty acid polyethylene glycol, POE hydrogenated lanolin alcohol ether, and the like.

Examples of the hydrocarbon oils that may be used may include liquid paraffin, ozocerite, squalane, pristane, paraffin, ceresin. squalene, vaseline, microcrystalline wax, isododecane, isohexadecane, and the like.

Examples of the higher fatty acid that may be used may include lauric acid, myristic acid, palmitic acid, stearic acid, behenic acid, oleic acid, undecylenic acid, tallic acid, isostearic acid, linoleic acid, linolenic acid, eicosapentaenoic acid (EPA), docosahexaenoic acid (DHA) and the like.

Examples of the higher alcohol that may be used may include linear alcohol (such as lauryl alcohol, cetyl alcohol, stearyl alcohol, behenyl alcohol, myristyl alcohol, oleyl alcohol, and cetostearyl alcohol); branched-chain alcohol (such as monostearylglycerin ether (batyl alcohol), 2-decyltetradecinol, lanolin alcohol, cholesterol, phytosterol, hexyldodecanol, isostearyl alcohol, and octyldodecanol) and the like.

Examples of the synthesis ester oils that may be used may include isopropyl myristate, cetyl octanoate, octyldodecyl myristate, isopropyl palmitate, butyl stearate, hexyl laurate, myristyl myristate, decyl oleate, hexyldecyl dimethyl octanoate, cetyl lactate, myristyl lactate, lanolin acetate, isocetyl stearate, isocetyl isostearate, cholesteryl 12-hydroxy stearate, ethylene glycol di-2-ethyl hexanoate, di-penta erythritol fatty acid ester, N-alkyl glycol monoisostearate, neopentyl glycol dicaprate, diisostearyl malate, glyceryl di-2-heptyl undecanoate, trimethyrol propane tri-2-ethyl hexanoate, trimethyrol propane triisostearate, pentaerythritol tetra-2-ethyl hexanoate, glyceryl tri-2-ethyl hexanoate, glyceryl trioctanoate, glyceryl triisopalmitate, trimethyrol propane triisostearate, cetyl 2-ethylhexanoate, 2-ethylhexyl palmitate, glyceryl trimyristate, glyceride tri-2-heptyl undecanoate, castor oil fatty acid methyl ester, oleyl oleate, acetoglyceride, 2-heptylundecyl palmitate, diisobutyl adipate, N-lauroyl-L-glutamic acid-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, triethyl citrate, and the like.

Examples of the silicone oil may include silicone compounds such as dimethylpolysiloxane, methylhydrogenpolysiloxane, methylphenylpolysiloxane, stearoxymethylpolysiloxane, polyether-modified organopolysiloxane, fluoroalkyl/polyoxyalkylene co-modified organopolysiloxane, alkyl-modified organopolysiloxane, terminal-modified organopolysiloxane, fluorine-modified organopolysiloxane, amino-modified organopolysiloxane, silicone gel, acrylic silicone, trimethylsiloxysilicic acid, silicone RTV rubber and the like.

Examples of the ultraviolet light absorbers may include benzoic acid family ultraviolet light absorber (such as p-aminobenzoic acid (hereinafter abbreviated as PABA), PABA monoglycerine ester, N,N-dipropoxy PABA ethyl ester, N,N-diethoxy PABA ethyl ester, N,N-dimethyl PABA ethyl ester, N,N-dimethyl PABA butyl ester, N,N-dimethyl PABA ethyl ester, etc); anthranilic acid family ultraviolet light absorber (such as homomenthyl N-acetylanthranilate etc); salicylic acid family ultraviolet light absorber (such as amyl salicylate, menthyl salicylate, homomenthyl salicylate, octyl salicylate, phenyl salicylate, benzyl salicylate, p-isopropanolphenyl salicylate, etc); cinnamic acid family ultraviolet light absorber (such as octyl methoxycinnamate, ethyl 4-isopropylcinnamate, methyl 2,5-diisopropylcinnamate, ethyl 2,4-diisopropylcinnamate, methyl 2,4-diisopropylcinnamate, propyl p-methoxycinnamate, isopropyl p-methoxycinnamate, isoamyl p-methoxycinnamate, octyl p-methoxycinnamate (2-ethylhexyl p-methoxycinnamate), 2-ethoxyethyl p-methoxycinnamate, cyclohexyl p-methoxycinnamate, ethyl α-cyano-β-phenylcinnamate, 2-ethylhexyl α-cyano-β-phenylcinnamate, glyceryl mono-2-ethylhexanoyl-diparamethoxy cinnamate, etc); benzophenone family ultraviolet light absorber (such as 2,4-dihydroxybenzophenone, 2,2′-dihydroxy-4-methoxybenzophenone, 2,2′-dihydroxy-4,4′-dimethoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl-4′-phenyl-benzophenone-2-carboxylate, 2-hydroxy-4-n-octoxybenzophenone, 4-hydroxy-3-carboxybenzophenone, etc); 3-(4′-methylbenzylidene)-d,l-camphor and 3-benzylidene-d,l-camphor; 2-phenyl-5-methylbenzoxazol; 2,2′-hydroxy-5-methylphenylbenzotriazol, 2-(2′-hydroxy-5′-t-octylphenyl) benzotriazol, 2-(2′-hydroxy-5′-methylphenylbenzotriazol; dibenzalazine; dianisoylmethane; 4-methoxy-4′-t-butyldibenzoylmethane; 5-(3,3-dimethyl-2-norbornylidene)-3-pentane-2-one; dimorpholinopyridazinone; 2-ethylhexyl 2-cyano-3,3-diphenylacrylate; 2,4-bis-([4-(2-ethylhexyloxy)-2-hydroxy]-phenyl)-6-(4-methoxyphenyl)-(1,3,5)-triazine, and the like.

The content by percentage of the entirety of the oily component(s) relative to the mass of the oily cosmetic is preferably 40% by mass or greater. When the content of the entirety of the oily component(s) is 40% by mass or greater, quick-dryability and makeup durability of the oily cosmetic can be maintained. The content by percentage of the entirety of the oily component(s) relative to the mass of the oily cosmetic may be 45% by mass or greater, 50% by mass or greater, 55% by mass or greater, 60% by mass or greater, or 65% by mass or greater. The content by percentage of the entirety of the oily component(s) relative to the mass of the oily cosmetic is preferably 80% by mass or less, more preferably 75% by mass or less, even more preferably 70% by mass or less. When the content by percentage of the oily component(s) is, at any rate, 80% by mass or less, quick-dryability and makeup durability of the oily cosmetic can be maintained. The content by percentage of the entirety of the oily component(s) relative to the mass of the oily cosmetic may be 65% by mass or less, 60% by mass or less, or 55% by mass or less.

{Powder}

The oily cosmetic of the present disclosure may further include a powder. The term “powder” used in the present Description is synonymous with “powder body”. The powder is not particularly limited so long as it is generally usable for cosmetic purposes, for example. Examples include talc, kaolin, sericite (sericite), muscovite, phlogopite, synthetic mica, synthetic fluorphlogopite, lepidolite, biotite, calcined talc, calcined sericite, calcined muscovite, calcined phlogopite, vermiculite, magnesium carbonate, calcium carbonate, aluminum silicate, barium silicate, calcium silicate, magnesium silicate, strontium silicate, tungstic acid metal salts, magnesium, silica, zeolite, barium sulfate, calcined calcium sulfate (calcined gypsum), calcium phosphate, fluorapatite, hydroxyapatite, ceramic powder, metallic soap (for example, zinc myristate, calcium palmitate, aluminum stearate, etc.), photochromic titanium oxide (titanium dioxide sintered with iron oxide), reduced zinc oxide; organic powders (for example, silicone elastomer powder, silicone powder, silicone resin-coated silicone elastomer powder, polyamide resin powder (nylon powder), polyethylene powder, polystyrene powder, styrene-acrylic acid copolymer resin powder, benzoguanamine resin powder, polytetrafluoroethylene powder, cellulose powder, etc.); inorganic white pigments (for example, titanium dioxide, zinc oxide, etc.); inorganic red pigments (for example, iron oxide (bengala), iron titanate, etc.); inorganic brown pigments (for example, 7-iron oxide etc.); inorganic yellow pigments (for example, yellow iron oxide, yellow ocher, etc.); inorganic black pigments (for example, black iron oxide, low-order titanium oxide, etc.); inorganic violet pigments (for example, mango violet, cobalt violet, etc.); inorganic green pigments (for example, chromium oxide, chromium hydroxide, cobalt titanate, etc.); inorganic blue pigments (for example, ultramarine, Prussian blue, etc.); pearl pigments (for example, bismuth oxychloride, fish scale flake, mica titanium, iron oxide-coated mica titanium, low-order titanium oxide-coated mica titanium, photochromic mica titanium, those in which talc, glass, synthetic fluorphlogopite, silica, bismuth oxychloride, etc. is used instead of mica as the substrate, those in which low-order titanium oxide, colored titanium oxide, iron oxide, alumina, silica, zirconia, zinc oxide, cobalt oxide, aluminum, etc. in addition to titanium oxide are coated as the coating material, that in which resin particles are coated on the surface of pearl pigment as the functional pearl pigment (Japanese Unexamined Patent Publication No. H11-92688), that in which aluminum hydroxide particles are coated on the surface of pearl pigment (Japanese Unexamined Patent Publication No. 2002-146238), that in which zinc oxide particles are coated on the surface of pearl pigment (Japanese Unexamined Patent Publication No. 2003-261421), that in which barium sulfate particles are coated on the surface of pearl pigment (Japanese Unexamined Patent Publication No. 2003-61229), etc.); metal powder pigments (for example, aluminum powder, copper powder, etc.); organic pigments such as zirconium, barium, or aluminum lakes (for example, organic pigments such as Red No. 201, Red No. 202, Red No. 204, Red No. 205, Red No. 220, Red No. 226, Red No. 228, Red No. 405, Orange No. 203, Orange No. 204, Yellow No. 205, Yellow No. 401 and Blue No. 404; and Red No. 3. Red No. 104, Red No. 106, Red No. 227, Red No. 230, Red No. 401, Red No. 505, Orange No. 205, Yellow No. 4, Yellow No. 5, Yellow No. 202, Yellow No. 203, Green No, 3, Blue No. 1, etc.); and natural dyes (for example, chlorophyll, 0-carotene, etc.).

The properties of the powder—such as average particle size, specific surface area, specific gravity, particle shape, whether it is porous or non-porous, whether it is hydrophilic or hydrophobic, and whether it is subjected to surface treatment—can be selected as appropriate depending on the purpose thereof. For example, to improve the lipophilicity and oil absorbency of the powder, it is possible to use a powder (oil-absorbent powder) whose particle surface has been hydrophobized. The powder may be a mixture including different types of powders.

The content by percentage of the powder can be set as appropriate depending on the purpose of the oily cosmetic. The content by percentage of the powder relative to the mass of the oily cosmetic may be, for example, 5% by mass or greater, 10% by mass or greater, or 20% by mass or greater. The content by percentage of the powder relative to the mass of the oily cosmetic may also be, for example, 45% by mass or less, 40% by mass or less, or 35% by mass or less.

{Aqueous Phase}

The oily cosmetic of the present disclosure may further include water. To suppress makeup smudging caused by sweat, it is preferred that the content by percentage of water is low, and the content of water relative to the mass of the oily cosmetic is preferably 10% by mass or less, more preferably 5% by mass or less, even more preferably 3% by mass or less. It is preferred that the oily cosmetic includes substantially no water.

{Other Components}

The composition of the present disclosure may include, as appropriate and as necessary, other components, such as alcohols, sugars, esters, anionic surfactants, cationic surfactants, amphoteric surfactants, nonionic surfactants, thickeners, moisturizers, water-soluble polymers, film-forming agents, UV absorbers, metal ion sequestering agents, amino acids, organic amines, polymer emulsions, pH adjusters, skin nutrients, vitamins, antioxidants, antioxidant aids, perfumes, etc., in amounts that do not inhibit the effects of the present invention.

The oily cosmetic of the present disclosure can suppress sweat from forming beads even when sweat arises in regions where the cosmetic has been applied. The wettability in regions where the oily cosmetic has been applied increases, and thus, it is possible to promote diffusion and drying of sweat. This eliminates the need to wipe off sweat, thereby suppressing makeup smudging caused by wiping. It is also possible to suppress sweat from trickling down, thus suppressing makeup smudging caused by trickling-down of sweat. Aesthetic appearance can also be improved by preventing beads of sweat from arising.

Further, according to the cosmetic of the present disclosure, it is possible to suppress sliminess felt when the oily cosmetic is applied to the skin.

By blending makeup components into the oily cosmetic, the makeup itself can be provided with a sweat-diffusion function.

The oily cosmetic of the present disclosure has a swift finish when applied to the skin. Thus, it is possible to reduce the time required by the user for applying the cosmetic, and also offer a comfortable feel upon use.

There may be cases where it is impossible, or utterly impractical, to directly define the oily cosmetic of the present disclosure based on the composition and/or properties of the oily cosmetic. Considering such circumstances, it is useful to define the oily cosmetic of the present disclosure according to methods for production or methods for use, as described below.

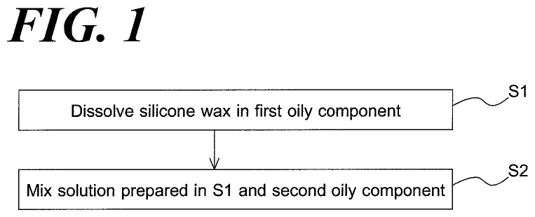

A second embodiment describes a method for producing the oily cosmetic according to the first embodiment of the present disclosure. FIG. 1 illustrates a schematic flowchart of a method for producing the oily cosmetic.

First, a hydrophilic silicone wax is dissolved in a first oily component (S1: dissolving step). The hydrophilic silicone wax is the aforementioned silicone wax. For the first oily component, it is possible to use a liquid-form UV absorber. For the UV absorber, it is possible to use, for example, a UV absorber including a benzene skeleton. Concrete examples may include 2-ethylhexyl 4-methoxycinnamate, octocrylene, diethylamino hydroxybenzoyl hexyl benzoate, butyl methoxydibenzoylmethane, ethylhexyl triazone, and bis-ethylhexyloxyphenol methoxyphenyl triazine. It is preferred to dissolve the silicone wax by adding it to a heated solvent.

The addition rate of the silicone wax in the dissolving step relative to the total mass of the silicone wax and the first oily component may be 5% by mass or greater, preferably 10% by mass or greater. The addition rate of the silicone wax in the dissolving step relative to the total mass of the silicone wax and the first oily component is preferably 80% by mass or less, preferably 75% by mass or less, even more preferably 70% by mass or less. If the content of the silicone wax exceeds 80% by mass, large amounts of silicone wax may remain undissolved.

Next, the solution obtained by dissolving the silicone wax is mixed with a second oily component including another oily component (S2: mixing step). In cases where the oily cosmetic is to include a powder, the powder is added to the oily component. The blending ratio among the components may be the same as the foregoing first embodiment. In this way, the oily cosmetic can be produced.

According to the oily cosmetic production method of the present disclosure, it is possible to prepare an oily cosmetic including a hydrophilic silicone wax, even in cases where the oily cosmetic does not include a water-based solvent. Also, the hydrophilic silicone wax can be dispersed uniformly in the oily cosmetic.

### EXAMPLES

The oily cosmetic and method for producing the same of the present disclosure will be described below according to examples. Note, however, that the oily cosmetic and method for producing the same of the present disclosure are not limited to the following examples. The unit employed for indicating the contents by percentage shown in the Tables is percent by mass (mass %).

### Test Examples 1 to 4

In Test Examples 1 and 2, oily cosmetics were prepared. In Test Examples 3 and 4, water-based cosmetics were prepared. A silicone wax was blended to the respective cosmetics of Test Examples 1 and 3. The prepared cosmetics were each tested for quick-dryability and makeup durability (lastingness of makeup). Table 1 shows the compositions and test results of the respective cosmetics. In the tables below, “proportion of volatile oily components” refers to the proportion of the volatile oily components to the entirety of the oily components.

In Test Examples 1 and 3, first, the silicone wax was dissolved in octyl methoxycinnamate (2-ethylhexyl 4-methoxycinnamate) while being heated. Next, the solution of the silicone wax was added to the other oily components and stirred. Then, the powders were added to the mixture. In this way, it was possible to prepare an oily cosmetic including silicone wax dispersed uniformly in the oily components. In Test Examples 3 and 4, the aqueous phase was emulsified in the oil phase.

{Quick-Dryability}

A 5-cm-square polymethyl methacrylate resin plate was coated with 2 μL/cm² of the respective cosmetic. The resin plate coated with the cosmetic was left standing at 37° C. for 5 minutes to let the cosmetic dry. Next, a drop (40 μL) of ion-exchanged water was dropped near the center of the surface coated with the cosmetic. After dropping, the plate was left at room temperature for 30 minutes, and the drying state of the dropped water was evaluated according to the following criteria. (Grades equal to or above C were judged as acceptable.) Note that the ion-exchanged water included a very small amount of water-soluble blue dye, and the test was designed so that the diffusion state (wetted state) of water on the makeup surface could be verified even after the water dried.

A: Water diffused the widest and dried completely.

B: Water diffused widely and dried completely.

C: Water diffused somewhat, and some water remained.

D: Water diffused slightly, but the dropped water remained.

E: Water did not diffuse, and almost all of the dropped water remained.

{Makeup Durability (Lastingness of Makeup)}

Ten expert panelists of cosmetics applied the aforementioned cosmetics on their skin and were asked to run and sweat. Then, three expert evaluators evaluated makeup durability, in terms of “runniness” and “oily shining”, on a 11-point scale according to the following evaluation criteria. (0 point: extremely poor makeup durability; 10 points: extremely good makeup durability). The average evaluation score was calculated and each cosmetic was evaluated as follows.

A: Average evaluation point was 8 points or higher.

B: Average evaluation point was 6 to 7 points.

C: Average evaluation point was 4 to 5 points.

D: Average evaluation point was 3 points or less.

Water dried up on the resin plates coated with the cosmetics of Test Examples 1 and 3 including the silicone wax. In contrast, water remained on the resin plates coated with the cosmetics of Test Examples 2 and 4 that did not include any silicone wax. These results suggest that, in Test Examples 1 and 3, the silicone wax improved wettability, which thus increased the surface area of water and promoted drying.

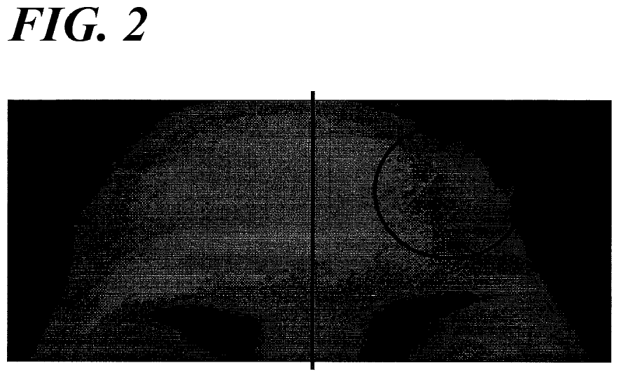

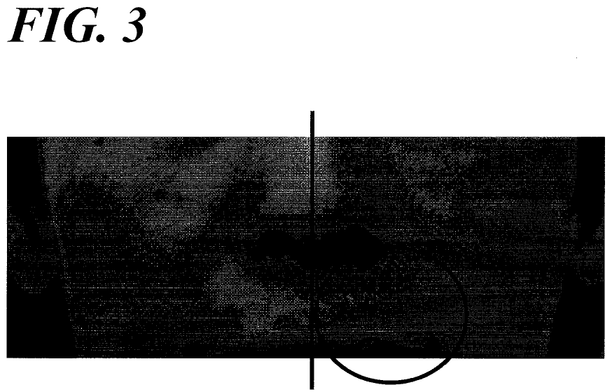

Further, a test subject who had actually applied the cosmetics of Test Examples 1 and 2 on his/her face, was asked to run and sweat. The state of sweat on each cosmetic was observed, to evaluate the appearance of sweat according to the following evaluation criteria. FIGS. 2 and 3 show photographs of the test subject's face in a sweating state. The left side of each photograph (i.e., the right side of the test subject's face) is the side applied with the cosmetic of Test Example 1, and the right side of each photograph (i.e., the left side of the test subject's face) is the side applied with the cosmetic of Test Example 2.

{Appearance (Shape) of Sweat}

A: Beads of water droplets are not observed.

B: Beads of water droplets are observed.

As illustrated in the section on the left side in FIGS. 2 and 3, no bead-like sweat was observed on the skin applied with the cosmetic of Test Example 1 including the silicone wax. In contrast, as indicated by the circles in the section on the right side in FIGS. 2 and 3, beads of sweat were observed on the skin applied with the cosmetic of Test Example 2 including no silicone wax, as in usual cases where sweating occurs. This shows that, by blending a silicone wax to the cosmetic, it is possible to improve wettability on the application surface, even on the skin, and thereby promote drying of sweat. Further, even when the user has sweated, it is possible to make the sweat less conspicuous and keep an appearance that looks as if the user has not sweated.

Skin applied with the cosmetics of Test Examples 3 and 4, which included an aqueous phase, obtained a poor evaluation in terms of makeup durability, and makeup smudging caused by sweating was observed. In contrast, skin applied with the cosmetics of Test Examples 1 and 2, which included no aqueous phase, obtained a good evaluation in terms of makeup durability, and there was little makeup smudging caused by sweat. These results show that, in order to suppress makeup smudging, it is preferred to blend a silicone wax in the oily cosmetic.

TABLE 1

| | Test Example | 1 | 2 | 3 | 4 |
|---|---|---|---|---|---|
| SW | Silicone wax *¹ | 10 | — | 10 | — |
| Volatile | Volatile silicone oil | 27.32 | 37.32 | 15 | 17.5 |
| oily | Isodecane | 4 | 4 | 4 | 6 |
| components | Isododecane | 4.5 | 4.5 | 4.5 | — |
| Non-volatile | Dimethicone (6CS) | 1.16 | 1.16 | 1.16 | 1.16 |
| oily | PEG-9 polydimethylsiloxyethyl | 1.5 | 1.5 | 1.5 | 1.5 |
| components | dimethicone | | | | |
| | Bis-butyldimethicone polyglyceryl-3 | 1 | 1 | 1 | 1 |
| | Isostearic acid | 1 | 1 | 1 | 1 |
| | Tocopherol | 0.02 | 0.02 | 0.02 | 0.02 |
| | Trimethylsiloxysilicate | 6 | 6 | 6 | 9 |
| | Trimethylsiloxysilicate/dimethiconol | 3 | 3 | 3 | — |
| | crosspolymer | | | | |
| | Octyl methoxycinnamate | 5 | 5 | 5 | 5 |
| | Isopropyl myristate | 2 | 2 | 2 | 2 |
| | Ethylparaben | 0.1 | 0.1 | 0.1 | 0.1 |
| Thickener | Disteardimonium hectorite | 0.5 | 0.5 | 0.5 | 0.5 |
| Powders | Hydrophobized titanium oxide fine | 18 | 18 | 18 | 18 |
| | particles | | | | |
| | Hydrophobized pigment-grade | 3 | 3 | 3 | 3 |
| | titanium oxide | | | | |
| | Hydrophobized red iron oxide | 0.86 | 0.86 | 0.86 | 0.86 |
| | Hydrophobized yellow iron oxide | 2 | 2 | 2 | 2 |
| | Hydrophobized black iron oxide | 0.04 | 0.04 | 0.04 | 0.04 |
| | Methyl methacrylate crosspolymer | 4 | 4 | 4 | 4 |
| | Silica | 2 | 2 | 2 | 2 |
| Aqueous | Ion-exchanged water | — | — | 11.87 | 21.87 |
| phase | EDTA-3Na · 2H₂O | — | — | 0.2 | 0.2 |
| | Citric acid | — | — | 0.02 | 0.02 |
| | Sodium citrate | — | — | 0.08 | 0.08 |
| | Ethanol | 3 | 3 | 3 | 3 |
| | Methylparaben | — | — | 0.15 | 0.15 |

| | | | | |
|---|---|---|---|---|
| Total | 100 | 100 | 100 | 100 |
| Total amount of volatile oily components (mass %) | 35.8 | 45.8 | 23.5 | 23.5 |
| Total amount of oily components (mass %) | 56.6 | 66.6 | 44.3 | 44.3 |
| Proportion of volatile oily components (%) | 63.3 | 68.8 | 53.1 | 53.1 |
| Quick-dryability | A | E | A | E |
| Makeup durability | A | A | C | C |
| Appearance of sweat | A | B | — | — |

*¹: Dow Corning (registered trademark) 2501 Cosmetic Wax from Dow Corning Toray Co., Ltd.

### Test Examples 5 to 11

Oily cosmetics were prepared by changing the content by percentage of the silicone wax, and quick-dryability and makeup durability were tested for the respective compositions, as in Test Examples 1 to 4. The evaluation criteria for quick-dryability and makeup durability are the same as those for Test Examples 1 to 4. Table 2 shows the compositions and test results.

Increasing the content by percentage of the silicone wax improved quick-dryability, but tended to deteriorate makeup durability. This thus suggests that the content by percentage of the silicone wax relative to the mass of the oily cosmetic is preferably 4% by mass or greater, more preferably 5% by mass or greater. This also suggests that the content by percentage of the silicone wax relative to the mass of the oily cosmetic is preferably 22% by mass or less, more preferably 20% by mass or less. For example, in cases where a cosmetic with better quick-dryability is desired, it is considered that the content by percentage of the silicone wax is preferably from 13 to 22% by mass, more preferably from 10 to 20% by mass. In cases where further suppression of makeup smudging is desired, it is considered that the content by percentage of the silicone wax is preferably from 4 to 12% by mass, more preferably from 5 to 10% by mass.

TABLE 2

| | | Test Example | | | | | | |
|---|---|---|---|---|---|---|---|---|
| | | 5 | 6 | 7 | 8 | 9 | 10 | 11 |
| SW | Silicone wax ^{*1} | 0 | 3 | 5 | 10 | 15 | 20 | 25 |
| Volatile | Volatile silicone oil | 33.33 | 31.33 | 30.33 | 27.33 | 22.33 | 19.33 | 14.33 |
| oily | Isodecane | 4 | 4 | 4 | 4 | 4 | 4 | 4 |
| components | Isododecane | 4.5 | 4.5 | 4.5 | 4.5 | 4.5 | 4.5 | 4.5 |
| Non- | Dimethicone (6CS) | 5 | 4 | 3 | 1 | — | — | — |
| volatile | PEG-9 polydimethylsiloxyethyl | 1.5 | 1.5 | 1.5 | 1.5 | 1.5 | 1.5 | 1.5 |
| oily | dimethicone | | | | | | | |
| components | Bis-butyldimethicone polyglyceryl-3 | 1 | 1 | 1 | 1 | 1 | 1 | 1 |
| | Isostearic acid | 1 | 1 | 1 | 1 | 1 | 1 | 1 |
| | Tocopherol | 0.02 | 0.02 | 0.02 | 0.02 | 0.02 | 0.02 | 0.02 |
| | Trimethylsiloxysilicate | 6 | 6 | 6 | 6 | 6 | 6 | 6 |
| | Trimethylsiloxysilicate/dimethiconol | 3 | 3 | 3 | 3 | 3 | 3 | 3 |
| | crosspolymer | | | | | | | |
| | Octyl methoxycinnamate | 0.5 | 0.5 | 1 | 5 | 5 | 5 | 5 |
| | Isopropyl myristate | 2 | 2 | 2 | 2 | 2 | 2 | 2 |
| | Ethylparaben | 0.1 | 0.1 | 0.1 | 0.1 | 0.1 | 0.1 | 0.1 |
| | Perfume | 0.15 | 0.15 | 0.15 | 0.15 | 0.15 | 0.15 | 0.15 |
| Thickener | Disteardimonium hectorite | 0.5 | 0.5 | 0.5 | 0.5 | 0.5 | 0.5 | 0.5 |
| Powders | Hydrophobized titanium oxide | 18 | 18 | 18 | 18 | 18 | 18 | 18 |
| | fine particles | | | | | | | |
| | Hydrophobized pigment-grade | 3 | 3 | 3 | 3 | 3 | 3 | 3 |
| | titanium oxide | | | | | | | |
| | Hydrophobized red iron oxide | 0.86 | 0.86 | 0.86 | 0.86 | 0.86 | 0.86 | 0.86 |
| | Hydrophobized yellow iron oxide | 2 | 2 | 2 | 2 | 2 | 2 | 2 |
| | Hydrophobized black iron oxide | 0.04 | 0.04 | 0.04 | 0.04 | 0.04 | 0.04 | 0.04 |
| | Methyl methacrylate | 4 | 4 | 4 | 4 | 4 | 4 | 4 |
| | crosspolymer | | | | | | | |
| | Silica | 2 | 2 | 2 | 2 | 2 | 2 | 2 |
| Alcohol | Ethanol | 3 | 3 | 3 | 3 | 3 | 3 | 3 |

| | | | | | | | |
|---|---|---|---|---|---|---|---|
| Total | 100 | 100 | 100 | 100 | 100 | 100 | 100 |
| Total amount of volatile oily components | 41.8 | 39.8 | 38.8 | 35.8 | 30.8 | 27.8 | 22.8 |
| (mass %) | | | | | | | |
| Total amount of oily components (mass %) | 62.1 | 59.1 | 57.6 | 56.6 | 50.6 | 47.6 | 42.6 |
| Proportion of volatile oily components (%) | 67.4 | 67.4 | 67.4 | 63.3 | 60.9 | 58.5 | 53.6 |
| Quick-dryability | E | D | B | B | A | A | A |
| Makeup durability | A | A | A | A | B | B | C |

### Test Examples 12 to 18

Oily cosmetics were prepared by changing the content by percentage of the volatile oily components, and quick-dryability and makeup durability were tested for the respective compositions, as in Test Examples 1 to 4. In addition to the evaluation items described in Test Examples 1 to 4, “swift finish” indicating usability was also adopted as an evaluation item. The evaluation criteria for usability are shown below. The evaluation criteria for quick-dryability and makeup durability are the same as those for Test Examples 1 to 4. Table 3 shows the compositions and test results.

{Usability (Swift Finish)}

A: Uniform application was possible when the cosmetic was rubbed into the skin.

B: Drying of sample was somewhat slow and application was somewhat uneven when the cosmetic was rubbed into the skin.

C: Drying of sample was slow and uniform application was not possible when the cosmetic was rubbed into the skin.

Reducing the content by percentage of the volatile oily components in the oily cosmetic tended to promote makeup smudging and also deteriorate quick-dryability. When the oily cosmetic of each Test Example is applied to the skin, the volatile oily components volatilize; thus, regions where the volatile oily components were present are likely to form voids, and sweat permeates through these voids. Also, the silicone wax dispersed in the oily cosmetic increases wettability. These are considered the reasons why sweat does not form beads but rather spreads on the skin. Test Example 18—which had a low content by percentage of the volatile oily components—suggests that the passages of sweat from the skin to the makeup surface diminished, thus leading to deterioration in quick-dryability and makeup durability. Further, reduction of the content by percentage of the volatile oily components caused a relative increase in the amount non-volatile components, which thus caused deterioration in usability. These results thus suggest that the content by percentage of the volatile oily components relative to the mass of the oily cosmetic is preferably 25% by mass or greater, more preferably 28% by mass or greater.

Test Examples 12 to 17 were able to obtain satisfactory results in terms of both quick-dryability and makeup durability. Thus, the content by percentage of the volatile oily components relative to the mass of the oily cosmetic may, at any rate, be 50% by mass or less. However, an increase in volatile oily components tended to deteriorate usability. This thus suggests that, to improve usability in addition to quick-dryability and makeup durability, it is preferred that the content by percentage of the volatile oily components relative to the mass of the oily cosmetic is preferably 48% by mass or less, more preferably 46% by mass or less, more preferably 44% by mass or less, even more preferably 42% by mass or less.

The results suggest that the content by percentage of all the oily components relative to the mass of the oily cosmetic is preferably 40% by mass or greater. The results also suggest that the content by percentage of all the oily components relative to the mass of the oily cosmetic is preferably 80% by mass or less, more preferably 75% by mass or less, even more preferably 70% by mass or less.

TABLE 3

| | | Test Example | | | | | | |
|---|---|---|---|---|---|---|---|---|
| | | 12 | 13 | 14 | 15 | 16 | 17 | 18 |
| SW | Silicone wax ^{*1} | 10 | 10 | 10 | 10 | 10 | 10 | 10 |
| Volatile | Volatile silicone oil | 40.26 | 36.78 | 32.80 | 27.33 | 21.36 | 17.88 | 15.39 |
| oily | Isodecane | 4 | 4 | 4 | 4 | 4 | 4 | 4 |
| components | Isododecane | 4.5 | 4.5 | 4.5 | 4.5 | 4.5 | 4.5 | 4.5 |
| Non-volatile oily components | Dimethicone (6CS) | 9 | 6.5 | 4.5 | 1 | — | — | — |
| | PEG-9 polydimethylsiloxyethyl | 1.5 | 1.5 | 1.5 | 1.5 | 1.5 | 1.5 | 1.5 |
| | dimethicone | | | | | | | |
| | Bis-butyldimethicone polyglyceryl-3 | 1 | 1 | 1 | 1 | 1 | 1 | 1 |
| | Isostearic acid | 1 | 1 | 1 | 1 | 1 | 1 | 1 |
| | Tocopherol | 0.02 | 0.02 | 0.02 | 0.02 | 0.02 | 0.02 | 0.02 |
| | Trimethylsiloxysilicate | 6 | 6 | 6 | 6 | 6 | 6 | 6 |
| | Trimethylsiloxysilicate/dimethiconol | 3 | 3 | 3 | 3 | 3 | 3 | 3 |
| | crosspolymer | | | | | | | |
| | Octyl methoxycinnamate | 5 | 5 | 5 | 5 | 5 | 2.5 | 2 |
| | Isopropyl myristate | 2 | 2 | 2 | 2 | — | — | — |
| | Ethylparaben | 0.1 | 0.1 | 0.1 | 0.1 | 0.1 | 0.1 | 0.1 |
| | Perfume | 0.15 | 0.15 | 0.15 | 0.15 | 0.15 | 0.15 | 0.15 |
| Thickener | Disteardimonium hectorite | 0.5 | 0.5 | 0.5 | 0.5 | 0.5 | 0.5 | 0.5 |
| Powders | Hydrophobized titanium oxide fine | 5.4 | 9 | 12.6 | 18 | 23.4 | 27 | 28.8 |
| | particles | | | | | | | |
| | Hydrophobized pigment-grade | 0.9 | 1.5 | 2.1 | 3 | 3.9 | 4.5 | 4.8 |
| | titanium oxide | | | | | | | |
| | Hydrophobized red iron oxide | 0.26 | 0.43 | 0.60 | 0.86 | 1.12 | 1.29 | 1.38 |
| | Hydrophobized yellow iron oxide | 0.6 | 1 | 1.4 | 2 | 2.6 | 3 | 3.2 |
| | Hydrophobized black iron oxide | 0.01 | 0.02 | 0.03 | 0.04 | 0.05 | 0.06 | 0.06 |
| | Methyl methacrylate crosspolymer | 1.2 | 2 | 2.8 | 4 | 5.2 | 6 | 6.4 |
| | Silica | 0.6 | 1 | 1.4 | 2 | 2.6 | 3 | 3.2 |
| Alcohol | Ethanol | 3 | 3 | 3 | 3 | 3 | 3 | 3 |

| | | | | | | | |
|---|---|---|---|---|---|---|---|
| Total | 100 | 100 | 100 | 100 | 100 | 100 | 100 |
| Total amount of volatile oily components (mass %) | 48.8 | 45.3 | 41.3 | 35.8 | 29.9 | 26.4 | 23.9 |
| Total amount of oily components (mass %) | 77.5 | 71.6 | 65.6 | 56.6 | 47.6 | 41.7 | 38.7 |
| Proportion of volatile oily components (%) | 62.9 | 63.3 | 63.0 | 63.3 | 62.7 | 63.3 | 61.8 |
| Quick-dryability | B | B | B | B | B | B | C |
| Makeup durability | A | A | A | A | A | A | C |
| Usability (Swift Finish) | C | B | A | A | A | A | C |

### Test Examples 19 to 24

A test was conducted to check how the effects are influenced by changing the proportion of the volatile oily components within the oily components. The evaluation tests and evaluation criteria are the same as those for the aforementioned Test Examples. Table 4 shows the compositions and test results.

All of Test Examples 19 to 24 exhibited sufficient quick-dryability. However, with a decrease in the proportion of the volatile oily components, the results for all evaluation items tended to deteriorate. A conceivable cause therefor is the diminishing of sweat passages, as described above. Further, Test Example 24 suggests that a relative increase of the non-volatile oily components resulted in deterioration of swift finish upon use. The results thus suggest that the proportion of the volatile oily components within the oily components is preferably 46% by mass or greater, more preferably 48% by mass or greater, more preferably 50% by mass or greater, more preferably 52% by mass or greater, even more preferably 54% by mass or greater. The results also show that quick-dryability and good makeup durability can be achieved when the proportion of the volatile oily components within the oily components is, at any rate, 80% by mass or less, preferably 75% by mass or less.

On the other hand, in terms of usability, dryability decelerated and deteriorated when the proportion of the volatile oily components within the oily components was too high. This thus suggests that, to improve usability, the proportion of the volatile oily components within the oily components is preferably 74% by mass or less, more preferably 70% by mass or less, even more preferably 65% by mass or less.

TABLE 4

| | | Test Example | | | | | |
|---|---|---|---|---|---|---|---|
| | | 19 | 20 | 21 | 22 | 23 | 24 |
| SW | Silicone wax ^{*1} | 10 | 10 | 10 | 10 | 10 | 10 |
| Volatile | Volatile silicone oil | 33.83 | 33.33 | 27.33 | 22.33 | 19.83 | 17.33 |
| oily | Isodecane | 4 | 4 | 4 | 4 | 4 | 4 |
| components | Isododecane | 4.5 | 4.5 | 4.5 | 4.5 | 4.5 | 4.5 |
| Non-volatile oily components | Dimethicone (6CS) | 1 | 1 | 1 | 6 | 8.5 | 11 |
| | PEG-9 polydimethylsiloxyethyl | 1.5 | 1.5 | 1.5 | 1.5 | 1.5 | 1.5 |
| | dimethicone | | | | | | |
| | Bis-butyldimethicone polyglyceryl-3 | 1 | 1 | 1 | 1 | 1 | 1 |
| | Isostearic acid | 1 | 1 | 1 | 1 | 1 | 1 |
| | Tocopherol | 0.02 | 0.02 | 0.02 | 0.02 | 0.02 | 0.02 |
| | Trimethylsiloxysilicate | 6 | 6 | 6 | 6 | 6 | 6 |
| | Trimethylsiloxysilicate/dimethiconol | 3 | 3 | 3 | 3 | 3 | 3 |
| | crosspolymer | | | | | | |
| | Octyl methoxycinnamate | 0.5 | 1 | 5 | 5 | 5 | 5 |
| | Isopropyl myristate | — | — | 2 | 2 | 2 | 2 |
| | Ethylparaben | 0.1 | 0.1 | 0.1 | 0.1 | 0.1 | 0.1 |
| | Perfume | 0.15 | 0.15 | 0.15 | 0.15 | 0.15 | 0.15 |
| Thickener | Disteardimonium hectorite | 0.5 | 0.5 | 0.5 | 0.5 | 0.5 | 0.5 |
| Powders | Hydrophobized titanium oxide fine | 18 | 18 | 18 | 18 | 18 | 18 |
| | particles | | | | | | |
| | Hydrophobized pigment-grade | 3 | 3 | 3 | 3 | 3 | 3 |
| | titanium oxide | | | | | | |
| | Hydrophobized red iron oxide | 0.86 | 0.86 | 0.86 | 0.86 | 0.86 | 0.86 |
| | Hydrophobized yellow iron oxide | 2 | 2 | 2 | 2 | 2 | 2 |
| | Hydrophobized black iron oxide | 0.04 | 0.04 | 0.04 | 0.04 | 0.04 | 0.04 |
| | Methyl methacrylate crosspolymer | 4 | 4 | 4 | 4 | 4 | 4 |
| | Silica | 2 | 2 | 2 | 2 | 2 | 2 |
| Alcohol | Ethanol | 3 | 3 | 3 | 3 | 3 | 3 |

| | | | | | | |
|---|---|---|---|---|---|---|
| Total | 100 | 100 | 100 | 100 | 100 | 100 |
| Total amount of volatile oily components (mass %) | 42.3 | 41.8 | 35.8 | 30.8 | 28.3 | 25.8 |
| Total amount of oily components (mass %) | 56.6 | 56.6 | 56.6 | 56.6 | 56.6 | 56.6 |
| Proportion of volatile oily components (%) | 74.8 | 73.9 | 63.3 | 54.5 | 50.1 | 45.6 |
| Quick-dryability | B | B | B | B | B | C |
| Makeup durability | A | A | A | A | B | C |
| Usability (Swift Finish) | C | B | A | A | B | C |

The oily cosmetic and method for producing the same of the present disclosure have been described according to the foregoing embodiments and examples, but they are not limited to the foregoing embodiments and examples and may encompass various transformations, modifications, and improvements made to the various disclosed elements (including elements disclosed in the Claims, Description, and Drawings) within the scope of the invention and according to the fundamental technical idea of the invention. Further, various combinations, substitutions, and selections of the various disclosed elements are possible within the scope of the claims of the invention.

Further issues, objectives, and embodiments (including modifications) of the invention are revealed also from the entire disclosure of the invention including the Claims.

The numerical ranges disclosed herein are to be construed in such a manner that arbitrary numerical values and ranges falling within the disclosed ranges are treated as being concretely described herein, even where not specifically stated.

### INDUSTRIAL APPLICABILITY

The oily cosmetic of the present disclosure can be used as a cosmetic to be applied to sweating regions, and particularly, can suitably be used in regions where makeup is applied, such as the face. Further, the oily cosmetic of the present disclosure can suitably be used at times and places where sweating is likely, such as during the summertime, when going to hot and humid locations, or when exercising or playing sports.

1. An oily cosmetic comprising:
4 to 22% by mass of a polyether-modified silicone wax;
40 to 80% by mass of an oily component, and
wherein: at least 46% by mass of said oily component is a volatile oily component, and
a content by percentage of said volatile oily component is at least 25% by mass relative to the mass of the oily cosmetic.

2. The oily cosmetic according to claim 1, wherein:
said volatile oily component includes a silicone oil.

3. The oily cosmetic according to claim 1, further comprising:
10 to 40% by mass of a powder relative to the mass of the oily cosmetic.

4. The oily cosmetic according to claim 3, wherein:
said powder includes an oil-absorbent powder.

5. The oily cosmetic according to claim 1, wherein:
a content by percentage of water is 5% by mass or less.

6. The oily cosmetic according to claim 1, wherein:
said polyether-modified silicone wax includes a compound represented by Chem. 1 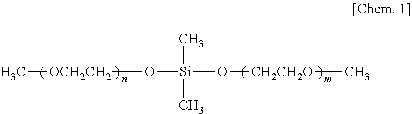
wherein m and n each independently represent an integer from 10 to 24.

7. The oily cosmetic according to claim 1, wherein:
said polyether-modified silicone wax includes bis-PEG-18 methyl ether dimethyl silane.

8. The oily cosmetic according claim 1, wherein:
said oily component includes at least one of an ester oil and/or a non-volatile silicone oil.

9. The oily cosmetic according to claim 1, wherein;
said oily component includes a UV absorber.

10. The oily cosmetic according to claim 1, wherein:
the proportion of said volatile oily component within the oily component is 48 to 74% by mass relative to the mass of the oily component; and
the content by percentage of said volatile oily component is 28 to 46% by mass relative to the mass of the oily cosmetic.

11. An oily cosmetic production method, comprising the steps of:
dissolving a hydrophilic polyether-modified silicone wax in a first oily component to prepare a solution; and
mixing said solution and a second oily component.

12. The production method, according to claim 11, wherein:
said first oily component includes a UV absorber.

13. The production method, according to claim 11, wherein:
said first oily component includes at least one compound selected from the group consisting of 2-ethylhexyl 4-methoxycinnamate, octocrylene, diethylamino hydroxybenzoyl hexyl benzoate, butyl methoxydibenzoylmethane, ethylhexyl triazone, and bis-ethylhexyloxyphenol methoxyphenyl triazine.

14. The production method, according to claim 11, wherein:
said second oily component includes a volatile oily component.

15. The production method, according to claim 14, wherein:
said volatile oily component includes a silicone oil.

16. The production method, according to claim 11, wherein:
said polyether-modified silicone wax includes a compound represented by Chem. 2: 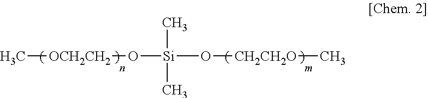
wherein m and n each independently represent an integer from 10 to 24.

17. The production method, according to claim 16, wherein:
said polyether-modified silicone wax includes bis-PEG-18 methyl ether dimethyl silane.
